(19) United States
(12) Patent Application Publication
(10) Pub. No.: US 2014/0378746 A1
(43) Pub. Date: Dec. 25, 2014

(54) ARTIFICIAL SPHINCTER

(71) Applicants: Mahdi Mohammadi, Orono, ME (US); Mohsen Shahinpoor, Bangor, ME (US); Krishna Mohan Bhatta, Skowhegan, ME (US)

(72) Inventors: Mahdi Mohammadi, Orono, ME (US); Mohsen Shahinpoor, Bangor, ME (US); Krishna Mohan Bhatta, Skowhegan, ME (US)

(21) Appl. No.: 14/309,655

(22) Filed: Jun. 19, 2014

Related U.S. Application Data
(60) Provisional application No. 61/837,038, filed on Jun. 19, 2013.

(51) Int. Cl. A61F 2/00

(52) U.S. Cl. CPC A61F 2/0036
USPC 600/30

(57) ABSTRACT

An implantable artificial sphincter for installation onto a duct of an organ in an animal body comprising an elongate tube having a first end and a second end, and having a magnet moveable therein between the first end and the second end; a metallic part located adjacent one end; and a flexible loop extending through the other end and affixed to the internal magnet.

### CROSS REFERENCE TO RELATED APPLICATION

This application claims priority from U.S. Provisional Application Ser. No. 61/837,038, filed Jun. 19, 2013, the contents of which are incorporated herein by reference.

### STATEMENT OF GOVERNMENT INTEREST

Not applicable.

### FIELD OF THE INVENTION

The present invention relates to medical devices. More particularly, the invention relates to implants acting as sphincters in human beings and animals. The invention has particular utility for use as urinary sphincters and will be described in connection with such utility, although other utilities are contemplated.

### BACKGROUND OF THE INVENTION

Urinary incontinence is the involuntary leakage of urine; in simple terms, to wee when not intend to. It is the inability to hold urine in the bladder because voluntary control over the urinary sphincter is either lost or weakened.

Urinary incontinence is a much more common problem than most people realize. In the United Kingdom it is estimated that at any one time at least 3 million people—5% of the total population—suffer from urinary incontinence. The US Department of Health and Human Services estimates that approximately 13 million Americans suffer from urinary incontinence.

Treatment for urinary incontinence depends on the type of incontinence, the severity of the problem and the underlying cause. In most cases, doctors suggest the least invasive treatments first, such as behavioral techniques and physical therapy, and move on to other options only if these techniques fail. Behavioral techniques include bladder training; Scheduled toilet trips, Fluid and diet management, and physical therapies include pelvic floor muscle exercises and electrical stimulation. Often, medications are used in conjunction with behavioral techniques.

There are also some medical devices available such as urethral inserts that is small tampon-like disposable device inserted into the urethra and acts as a plug to prevent leakage.

If other treatments aren't working, several surgical procedures have been developed to fix problems that cause urinary incontinence. These surgeries include: sling procedures, bladder neck suspension, and artificial urinary sphincter.

It is a well known problem in men especially after removal of prostate for cancer of prostate and the current invention will be most suitable for these subgroup of patients. A patient who uses 1-3 pads a day for their incontinence is usually advised not to have the currently available artificial sphincters. This is because the surgical procedure currently available requires placement of fluid reservoirs inside the abdomen (pelvis) and a manual dexterity to be able to pump fluid in and out of this reservoir through a valve housed in the scrotum.

Currently available urinary sphincter devices are multi-component and cumbersome to place surgically in the human body. Multiple components need to be installed in different locations, and this is a time consuming and difficult process. Malfunctions in these devices are generally very difficult to detect and correct. They have very complicated components, making assembly, difficult and prone to complications and failure. Those devices which use an inflatable ring to close off the urethra are prone to non-uniform inflation and resultant injury to the urethra. Others may not be effective for severe incontinence, or require access through the skin to operate. Some employ a magnetically operated valve requiring a specialized external magnetic key to operate, the key being subject to loss or misplacement. Devices inserted directly into the urethra increase the likelihood of infection. Likely failure modes in many devices leave the urethra closed, which would result in the need of timely surgery to avoid bladder damage.

U.S. Pat. No. 3,810,259, issued May 14, 1974, to Summers is complicated and intrusive, and requires the use of magnetic keys which are subject to loss, or may otherwise be unavailable.

U.S. Pat. No. 5,643,194, issued Jul. 1, 1997, to Negre is similarly subject to loss of the very specialized magnetic key, and is primarily directed toward relief and drainage of fluid for treatment of hydrocephalus. This device could not be used to control urinary incontinence where a urethra remains intact.

U.S. Pat. No. 4,571,749, issued Feb. 25, 1986, and U.S. Pat. No. 4,784,660, issued Nov. 15, 1988, both to Fischell, describe inflatable cuffs located around the urethra. These devices oftentimes are not uniformly inflated, resulting in possible damage to the urethra.

U.S. Pat. No. 4,118,805, issued Oct. 10, 1978, to Reimelds describes an artificial sphincter. It is a very complicated device, and thus can be subject to failure or misadjustment.

U.S. Pat. No. 6,074,341, issued Jun. 13, 2000, to Anderson et al. describes mechanically complicated embodiments of an artificial urethra sphincter. The mechanical operation of the occlusive apparatus appears to be difficult to operate through the skin. Other means of operation are complicated and subject to failure.

U.S. Pat. No. 6,095,969, issued Aug. 1, 2000, to Karram et al. describes an implantable device for controlling stress incontinence in female patients. This device is intended to control incontinence when a patient is coughing, etc. It would not be effective in more severe cases of incontinence.

U.S. Pat. No. 6,063,119, issued May 16, 2000, to Pintauro et al. describes a device for maintaining urinary incontinence. The device is inserted into the urethra and partially into the bladder. Such a device enhances the likelihood of infections and may potentially cause damage to the urethra inner wall or bladder.

The foregoing discussion of the prior art derives primarily from U.S. Pat. No. 6,432,038 which describes a device for treatment of urinary incontinence including an outer cuff surrounding an inner cuff having interconnecting fluid-containing cuff chambers partially separated from each other by inner cuff walls. The device according to the '038 patent includes circumferential wires run over pulleys that are attached to a push button assembly which is located and attached to the outer overlap portion of the outer cup. The push button assembly contains a retractable device similar to that of a retractable ball point pen. Pushing the push button places tension on the wires which in turn compresses the fluid-containing portions so as to compress and shut the urethra. Upon pushing the button a second time, pressure is released. This device is relatively bulky and has a significant number of parts which could be prone to wear and/or failure, resulting in the need for a repeated invasive surgery.

Another prior art artificial sphincter which is currently implanted for men (U.S. Pat. No. 4,222,377) is a device made of silicone rubber that is used to treat urinary incontinence. This artificial sphincter has an inflatable cuff that fits around the urethra close to the point where it joins the bladder. A balloon regulates the pressure of the cuff, and a bulb controls inflation and deflation of the cuff. The balloon is surgically placed within the pelvic area, and the control pump is placed in the scrotum.

The cuff is inflated to keep urine from leaking. When urination is desired, the cuff is deflated, allowing urine to drain out.

The problem with this sphincter is that it needs a more invasive surgery to be implanted, including a pump in scrotum, a balloon reservoir in the pelvic area, a cuff around the urethra, and tubing between them. In addition, due to having a lot of parts, there is a higher chance for failure. If the device fails, or the cuff erodes, the surgery must be repeated. In a study published in 2001, 37% of women had the implant after an average of seven years, but 70% had the original or a replacement and 82% were continent. Studies on men report similar findings. Plus with this particular implant, the scrotum gets very sore because of pumping.

These problems were the motivations for quite a number of doctors and engineers to look for simpler and more reliable designs, and there are a lot of patents addressing the artificial urinary sphincters. Some of the new ideas focus on silicon rubber sphincter cuffs inflated by a fluid, operated either by hand pump or external magnet (U.S. Pat. No. 5,562,598).

The other category is the mechanical cuffs operated by pressing the cuff itself by hand (U.S. Pat. No. 5,888,188). Others used spring loaded cuffs for closing the urethra which are operated manually or magnetically (U.S. Pat. No. 6,409,656).

### SUMMARY OF THE INVENTION

The present invention provides an improvement over artificial sphincters such as described above. More particularly, the present invention provides an artificial sphincter which is based on a very simple and reliable design. There are only four parts: a tube which provides a housing for an internal magnet for axial movement, and also provides a bed for the lumen, in this case in the lower part of corpus spongiosum; an internal magnet which is connected to a flexible ribbon, and, as it moves up and down opens and closes the lumen, in this case the urethra; a ribbon which goes around the lumen, in this case the corpus spongiosum, and pushes it against the tube in a closed position; and a metallic part which is located at the end of the tube and keeps the lumen closed by magnetic attraction to the internal magnet.

### BRIEF DESCRIPTION OF THE DRAWINGS

Further features and advantages of the present invention will be seen from the following detailed descriptions, taking in conjunction with the accompanying drawings in which:

FIGS. 1 A and 1B are X-ray perspective views showing an artificial sphincter in accordance with the present invention in an open and in a closed position, respectively;

FIGS. 2A-2D are elevational views and cross-sectional views of the artificial sphincter of FIG. 1 in a closed position;

FIGS. 3A-3D are side elevational views and cross-sectional views of the artificial sphincter of claim 1 in an open position;

FIG. 4 is a side elevational view in cross section, showing the artificial sphincter in place in an open position;

FIG. 5 is a view similar to FIG. 4 showing the artificial sphincter in place in a closed position;

FIG. 6 is a view, similar to FIG. 4, showing how an external magnet is introduced and applied to the artificial sphincter in accordance with the present invention.

FIGS. 7A and 7B views are similar to FIGS. 1A and 1B, showing an alternative embodiment of the artificial sphincter in accordance with the present invention;

FIGS. 8A-8B are side and cross-sectional views of the alternative version of FIGS. 7A-7B in a closed position;

FIGS. 9A-9B are used similar to FIGS. 8A and 8B, showing the alternative version in an open position.

FIG. 10 is a side elevational view, in cross section, of a yet another embodiment of artificial sphincter in accordance with the present invention, shown in an open position; and

FIG. 11 is a view similar to FIG. 10, of the FIG. 10 alternative embodiment artificial sphincter in a closed position.

### DETAILED DESCRIPTION OF THE INVENTION

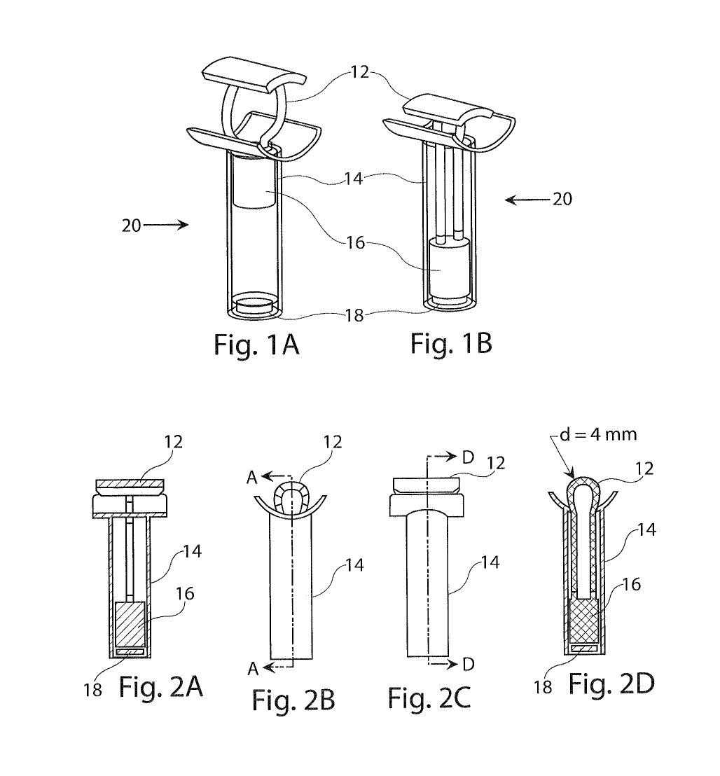

FIGS. 1A-1D show the 4 different parts of an artificial sphincter 20 in accordance with a first embodiment of the invention, in open and closed positions. These parts are a ribbon or loop 12, a tube 14, a magnet 16 and a metallic target or part 18. The tube is made of a biocompatible material. The magnet is carried in the tube and is capable of axially moving within the tube from one end to the other, pulling or releasing the sphinctering ribbon or string loop 3, which in turn is looped around a biological or non biological fluid duct or lumen. The device also has a metallic part which is located at one end of the tube to keep the duct or lumen selectively closed by attraction of the internal magnet.

FIG. 2 shows the side views and cut-out views of the sphincter in a closed position. Part 3 is the ribbon which is attached to the internal magnet. Part 14 is the tube containing the internal magnet 16 and the metallic target or part 18, which may take the form of a metal plate. In a closed position the internal magnet 16 is attracted to the metallic part 18. Tube 14 is formed of a biocompatable material such as polyvinylchloride (PVC) or Teflon (PTFE) or a non-magnetic biocompatible metal such as platinum or gold. Magnet 18 preferably is formed of a rare earth magnetic material such as a neodymuim magnet coated with a biocompatable material such as silicone rubber or PTFE. Ribbon or loop 12 is formed of a biocompatible synthetic fabric such as Gortex, a polyamide fiber such as nylon, or an acrylic fiber such as Orlon.

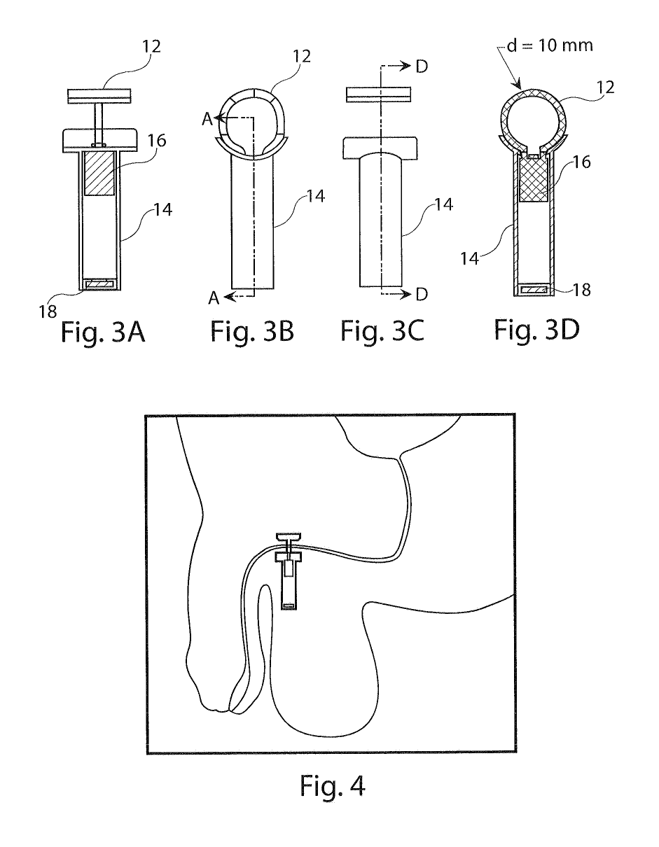

FIGS. 3A-3D show side views and cut-out views of the sphincter in an open position and FIG. 4 shows the implanted sphincter in an open position. Part 12 is the ribbon which is attached to the internal magnet, part 14 is the tube containing the internal magnet 16 and the metallic target or part 18. In this open position the internal magnet 16 is spaced away from the metallic target or part 18 and stays adjacent the other end of the tube 14 away from the metallic target or part 18, by the force of the sphincter due to internal pressure of fluid moving through the urethra. In use, the patient uses an external magnet 20 to repulse and move the internal magnet 16 upward in order to loosen the ribbon or loop 12 and release the sphincter and open the urethra, so that he or she can void.

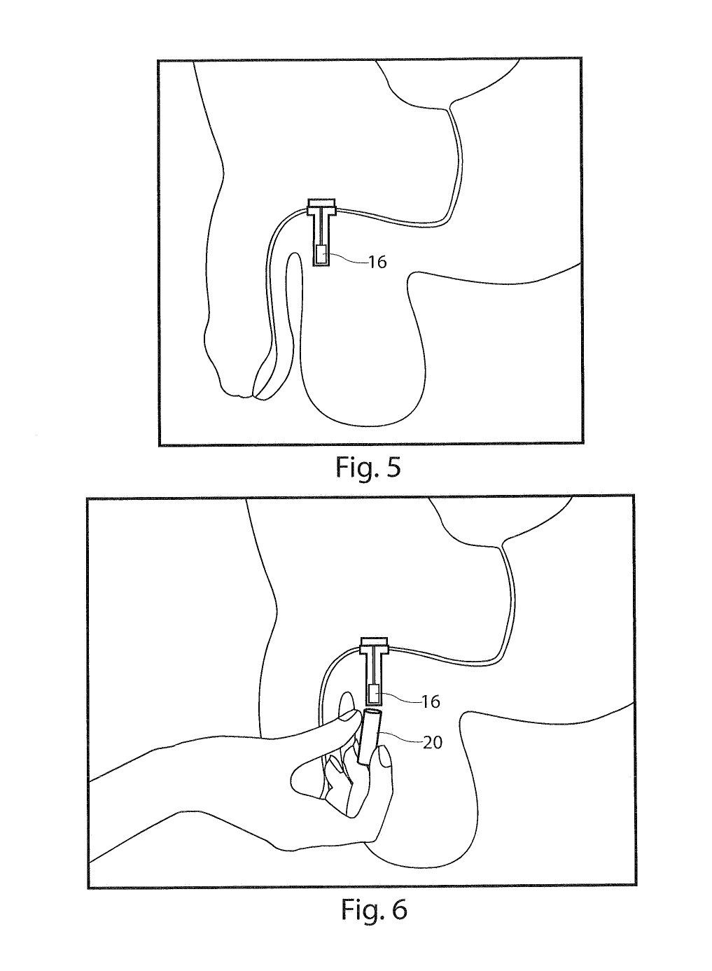

FIG. 5 shows the implanted sphincter in the closed position. Note that the ribbon is attached to the internal magnet and the tube contains the internal magnet 16 and the metallic part. In this closed position the internal magnet 16 is attracted and attached to the metallic part. The patient once again introduces the external magnet 20 in a reverse North-South configuration to the internal magnet (South-North) to pull the internal magnet 16 down towards the metallic part end and close the sphincter loop to stop fluid flow.

FIG. 6 shows how the external magnet 20 is to be introduced and applied to the urethral sphincter system. The patient uses an external magnet 20 to repulse and move the internal magnet 20 upward in order to make the loop loose and release the sphincter and open the urethra, so that he or she can void. Yet, the patient once again introduces the external magnet 20 in a reverse North-South configuration to the internal magnet (South-North) to pull the internal magnet 16 down towards the metallic target or part 18 and close the sphincter loop to stop fluid flow.

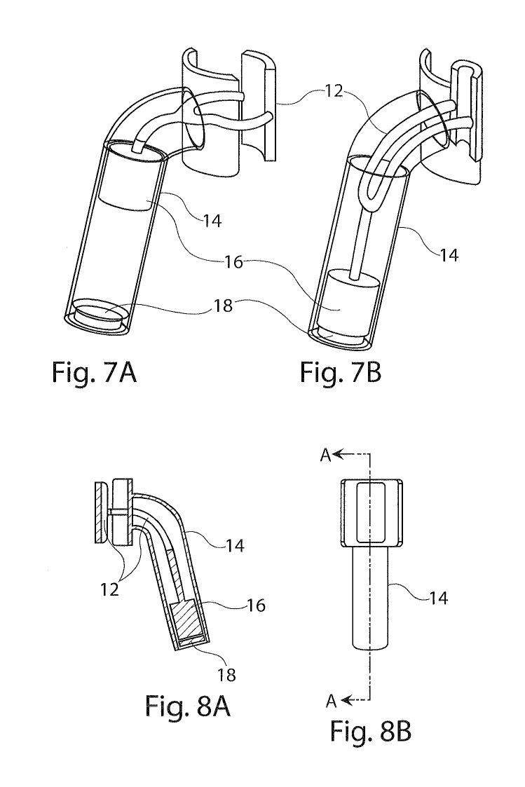

FIGS. 7A and 7B show the 4 different parts of the sphincter in open and closed positions for a curved version of the sphincter system. These parts are the ribbon or loop 12, the tube 14, the magnet 16 and the metallic magnet or part 18. As before, the tube 14 is made from a biocompatible material, the internal magnet 16 is capable of axially moving within the tube from one end to the other further pulling or releasing the sphinctering ribbon or loop 12, which in turn is looped around a biological or non biological fluid duct or lumen. The device also has a metallic part which is located at the end of the implanted tube to keep the duct or lumen closed by attracting the internal magnet 16.

FIGS. 8A and 8B show the side views and cut-out views of the sphincter in a closed position for a curved version of the sphincter system. Note that part 12 is the ribbon which is attached to the internal magnet 16, part 14 is the prismatic tube containing the internal magnet 16 and the metallic target or part 18. In this closed position the internal magnet 16 is attracted to the metallic target or part 18.

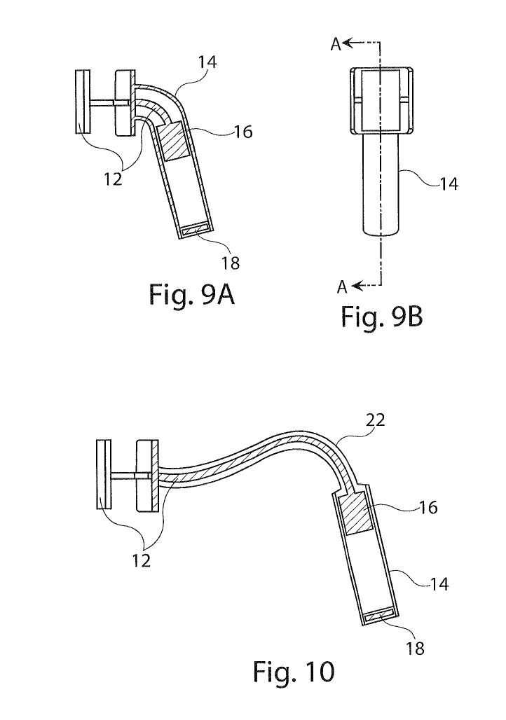

FIGS. 9A and 9B show the side views and cut-out views of the sphincter open position for a curved version of the sphincter system. As before, part 12 is the ribbon which is attached to the internal magnet 16, part 14 is the tube containing the internal magnet 16 and the metallic target or part 18. In this open position the internal magnet is away from the metallic target or part 18 and stays on the other end of the tube 14 away from the metallic target or part 18. As before, the patient uses an external magnet (not shown) to repulse and move the internal magnet 16 upward in order to make the loop 12 loose and release the sphincter and open the urethra, so that he or she can void.

FIG. 10 shows the artificial sphincter with a curved and flexible tubular extension 22 in open position. This tubular and curved tubular extension 22 is intended for internal organ duct at a distance. Part 14a shows the solid part of the tube in which the internal magnet 16 moves and part 22 depicts the flexible extension of the tube wherein the ribbon or loop 12 operates and connects to the sphincter loop. As before, part 12 is the ribbon or loop which is attached to the internal magnet 16, part 14 is the tube containing the internal magnet 16 and the metallic target or part 18. In this open position the internal magnet 16 is away from the metallic target or part 18.

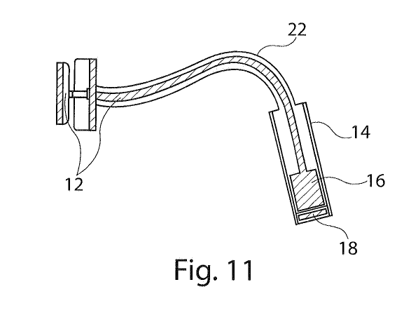

FIG. 11 shows the artificial sphincter with curved and flexible tubular extension 22 in closed position. This tubular and curved tubular extension 22 is intended for internal organ duct at a distance. Part 14 shows the solid part of the tube in which the internal magnet 16 moves and part 22 depicts the flexible extension of the tube wherein the ribbon 12 operates and connects to the sphincter loop. Note that part 12 is the ribbon or loop which is attached to the internal magnet, part 14 is the tube containing the internal magnet 16 and the metallic target or part 18. In this closed position the internal magnet 16 is attracted to the metallic target or part 18.

In accordance with a preferred embodiment, the artificial sphincter of the present invention is implanted in a man's scrotum as shown in FIG. 4 and FIG. 5. It provides the patient with easy, fast and reliable control on urination time for those who suffer from incontinence.

FIG. 4 shows the sphincter in open position, where the patient is urinating. During the urination, the sphincter remains open due to the urination pressure in the urethra which varies from around the 60 cm of water column at the beginning of the urination to zero at the end of urination. Even when the urination pressure goes to zero the sphincter still remains open, because the internal magnet 16 is away from the metallic target or part 18. At this point the patient introduces the closing end of the external magnet 22 (S pole) to the metallic target or part 18. The metallic target or part 18 enhances the magnetic field in the axial direction of the tube 14. Due to this magnetic field, the internal magnet 16 is attracted to the metallic target or part 18 and closes the urethra by pulling the ribbon or loop 12 down. This happens in milliseconds and then the patient removes the external magnet.

The internal magnet 16 stays in this position due to magnetic attraction to the metallic target or part 18 and it does not let any leakage even though the bladder starts building up a pressure again. The ribbon diameter around the corpus spongiosum in closed position is around 4 mm which can be adjusted for patients of different sizes by selecting the ribbon or loop starting point.

For opening the sphincter, the patient introduces the opening side of the external magnet 22 to the metallic target or part 18. By using an external magnet 22 which is much stronger than the internal magnet 16, the repulsive force of the external magnet 22 overcomes the magnetic attraction between the internal magnet 16 and the metallic target or part 18, and makes the internal magnet 16 move up in the tube. At this point the patient can start urinating and either remove the external magnet or let it stay connected to the metallic part on the scrotum skin (in men). The ribbon diameter around the corpus spongiosum in open position is around 10 mm which also can be adjusted for patients of different sizes by selecting the ribbon snapping point.

The external magnet can be a standard, off the shelf magnet. Preferably, the magnet is cylindrical (d=8 mm, 1=20 mm) or cubical (12×12×5 mm) or other shape, and can, if desired, be attached to the patient's underwear or attached to the lower part of the scrotum for convenience.

Thus, the present invention provides a simple implantable artificial sphincter having few moving parts. And, unlike prior art, magnetically operated sphincters which require a specialized external magnet keys, the present invention employs a conventional off-the-shelf magnet to activate the sphincter.

Various changes may be made in the invention without departing from the spirit and scope. For example, while the invention had been illustrated for treatment of male patients, the invention also may be employed with female patients by placing the sphincter around the female patient's urethra. The invention also may be used, for example, for controlling leakage from other body lumen tissues, for example, for controlling fecal incontinence, stoma or reflux, i.e. through the esophagus.

1. An implantable artificial sphincter for installation onto a duct of an organ in an animal body comprising:
an elongate tube having a first end and a second end, and having an internal magnet moveable therein between the first end and the second end;
a metallic part located adjacent one end; and
a flexible loop for looping around the duct, extending through the other end and affixed to the internal magnet.

2. The implantable artificial sphincter according to claim 1, further including an external magnet for controlling a position of the internal magnet in the tube.

3. The implantable artificial sphincter according to claim 2, wherein the diameter of the loop is adjusted by moving the internal magnet.

4. The implantable artificial sphincter according to claim 3, wherein the diameter of the loop is adjustable between about 5 mm to about 10 mm.

5. The implantable artificial sphincter according to claim 1, wherein the loop comprises a hollow tube filled with a fluid or hydrogel to create soft contact with the duct.

6. The implantable artificial sphincter according to claim 1, wherein the loop is encased or formed of a biocompatible material.

7. The implantable artificial sphincter according to claim 6, wherein the loop is formed of a synthetic fabric selected from the group consisting of a polyamide fiber, an acrylic fiber, Gortex and a PTFE fiber.

8. The implantable artificial sphincter according to claim 1, wherein the elongated tube has a curved and flexible extension for reaching any organ duct at a distance.

9. The implantable artificial sphincter according to claim 1, wherein the internal magnet is formed of a rare earth magnet.

10. The implantable artificial sphincter according to claim 8, wherein the rare earth magnet comprises a neodymium magnet coated with a biocompatible material.

11. A method for controlling flow through a duct in an animal body, comprising surgically implanting the artificial sphincter of claim 1 into the body of the animal, looping the flexible loop around the duct, and opening or closing the artificial sphincter by positioning a magnet external to the body adjacent the implanted sphincter.

12. The method of claim 10, wherein the animal is a human animal.

13. The method of claim 11, wherein the human animal is a male, and the duct is the urethra.

14. The method of claim 11, wherein the duct comprises the colon or esophagus.
